(19) United States
(12) Patent Application Publication
(10) Pub. No.: US 2026/0108700 A1
(43) Pub. Date: Apr. 23, 2026

(54) NEURO-ACOUSTIC VAGAL MODULATION SYSTEM, DEVICE, AND METHOD

(71) Applicant: Ezymind Healthcare Private Limited, Pune (IN)

(72) Inventors: Shalmali Kadu, Pune (IN); Mitansh Khurana, New Delhi (IN)

(21) Appl. No.: 19/362,353

(22) Filed: Oct. 18, 2025

(30) Foreign Application Priority Data
Oct. 21, 2024 (IN) 202421080053

(51) Int. Cl. A61M 21/02; A61M 21/00; A61N 1/36

(52) U.S. Cl. CPC A61M 21/02; A61N 1/36031; A61N 1/36036; A61M 2021/0027; A61M 2021/0072; A61M 2205/502; A61M 2230/65

(57) ABSTRACT

Disclosed is a system, a device, and a method for neuro-acoustic vagal modulation. The method includes the steps of receiving (706) a session type selection, determining (708) stimulation parameters, generating (710) stimulation signals, and transmitting (712) the signals to a neuro-acoustic vagal modulation device (102) positioned behind a user's ear. The method includes receiving (714) physiological response data from the device (102) or an auxiliary wearable device (108), dynamically adjusting (716) the stimulation parameters based on the received data, and transmitting (718) the adjusted signals to the device (102).

### TECHNICAL FIELD

The present disclosure relates to neuro-acoustic vagal modulation systems and devices, and more particularly to a neuro-acoustic vagal modulation system, device, and method for stress and anxiety relief through stimulation of the parasympathetic nervous system.

### BACKGROUND

The field of neuro-acoustic vagal modulation has gained significant attention in recent years as a potential non-invasive approach for managing stress, anxiety, and other neurological conditions. The technology combines electrical stimulation of specific nerve pathways with acoustic signals to modulate brain activity and influence physiological responses.

Conventional methods for stress and anxiety relief often rely on pharmaceutical interventions or traditional therapeutic techniques. While these approaches can be effective, they may have limitations such as side effects, dependency, or the need for ongoing professional intervention. Additionally, existing non-invasive neuromodulation devices typically focus on either electrical stimulation or acoustic stimulation separately, potentially limiting their overall effectiveness.

Current vagal modulation systems often lack personalization and real-time adaptability. Many devices use pre-set parameters that do not account for individual physiological differences or variations in a user's state throughout a session.

Furthermore, existing solutions may not provide accurate placement verification or comprehensive biofeedback integration, which can lead to suboptimal stimulation and reduced therapeutic outcomes.

Therefore, there exists a need for a technical solution that solves the aforementioned problems of conventional systems and methods for neuro-acoustic vagal modulation for stress and anxiety relief.

### SUMMARY

The summary is provided to introduce a selection of concepts in a simplified form that are further described below in the detailed description. The summary is not intended to identify key features or essential features of the claimed subject matter, nor is it intended to be used as an aid in determining the scope of the claimed subject matter.

In an aspect of the present disclosure, a neuro-acoustic vagal modulation system is disclosed. The system includes the neuro-acoustic vagal modulation device. The system includes a user device comprising a user interface configured to receive a session type selection from a user. The system includes an audio device configured to deliver audio signals to the user. The system includes an information processing apparatus comprising processing circuitry configured to receive the session type selection from the user device. The processing circuitry is configured to determine initial stimulation parameters based on the selected session type. The processing circuitry is configured to transmit the initial stimulation parameters to the neuro-acoustic vagal modulation device. The processing circuitry is configured to receive accelerometer data and GSR data from the neuro-acoustic vagal modulation device. The accelerometer data facilitates the processing circuitry to understand the amount of muscle fibres present underneath the surface where the neuro-acoustic vagal modulation device is placed and to understand the feedback dynamics of this segment on the user's body under stimulation. The processing circuitry is configured to dynamically adjust the stimulation parameters based on the received position data and GSR data. The processing circuitry is configured to transmit the adjusted stimulation parameters to the neuro-acoustic vagal modulation device.

In some aspects of the present disclosure, the processing circuitry may be further configured to analyze post-session data to evaluate effectiveness of the session. The processing circuitry may be further configured to provide smart nudges to the user via the user device suggesting additional sessions or professional support based on the analysis.

In some aspects of the present disclosure, the processing circuitry may be further configured to receive data from the auxiliary wearable device including at least one of heart rate, sleep time, wake-up time, activity levels, or heart rate variability (HRV).

The processing circuitry may be further configured to adjust the stimulation parameters based on the received data from the auxiliary wearable device.

In some aspects of the present disclosure, the processing circuitry may be further configured to calculate a Vagal Activity Index (VAI) based on the received data from the auxiliary wearable device and the GSR data from the neuro-acoustic vagal modulation device. The processing circuitry may be further configured to optimize the stimulation parameters based on the calculated VAI.

In some aspects of the present disclosure, the processing circuitry may be further configured to synchronize the electrical stimulation signals generated by the neuro-acoustic vagal modulation device with the audio signals delivered by the audio device. The processing circuitry may be further configured to dynamically adjust the synchronization based on the calculated VAI and the received position data.

In an aspect of the present disclosure, a method for neuro-acoustic vagal modulation is disclosed. The method includes receiving a selection of a session type from a user device by a processing circuitry. The processing circuitry determines stimulation parameters based on the selected session type. The processing circuitry generates stimulation signals based on the determined stimulation parameters. The processing circuitry transmits the stimulation signals to a neuro-acoustic vagal modulation device positioned behind a user's ear. The processing circuitry receives physiological response data from at least one of the neuro-acoustic vagal modulation device or an auxiliary wearable device. The processing circuitry dynamically adjusts the stimulation parameters based on the received physiological response data. The processing circuitry transmits the adjusted stimulation signals to the neuro-acoustic vagal modulation device for application to the user.

In some aspects of the present disclosure, the session type may be selected from a group consisting of sleep, relax, and focus.

In some aspects of the present disclosure, the physiological response data may comprise at least one of galvanic skin response (GSR), heart rate, or heart rate variability (HRV).

In some aspects of the present disclosure, the method may include verifying correct placement of the neuro-acoustic vagal modulation device using an accelerometer-based model by the processing circuitry.

In some aspects of the present disclosure, the method may include providing guidance to the user via the user device for correct placement of the neuro-acoustic vagal modulation device if incorrect placement is detected by the processing circuitry.

The foregoing general description of the illustrative aspects and the following detailed description thereof are merely exemplary aspects of the teachings of the disclosure and are not restrictive.

### BRIEF DESCRIPTION OF DRAWINGS

The following detailed description of the preferred aspects of the present disclosure will be better understood when read in conjunction with the appended drawings. The present disclosure is illustrated by way of example, and not limited by the accompanying figures, in which like references indicate similar elements.

FIG. 1 illustrates a block diagram of a neuro-acoustic vagal modulation system, according to an aspect of the present disclosure.

FIG. 2 illustrates a block diagram of a processing unit, according to an aspect of the present disclosure.

FIG. 3 illustrates a block diagram of an information processing apparatus for a neuro-acoustic vagal modulation system, according to an aspect of the present disclosure.

FIG. 4 illustrates a correctly placed neuro-acoustic vagal modulation device to stimulate the auricular branch and the pharyngeal branch of the vagus nerves, according to an aspect of the present disclosure.

FIG. 5 illustrates a representation of working of the GSR bio-feedback mechanism, according to an aspect of the present disclosure.

FIG. 6 illustrates a representation of working of the neuro-acoustic vagal modulation system, according to an aspect of the present disclosure.

FIG. 7 illustrates a flowchart depicting a method for neuro-acoustic vagal modulation via a device placed behind the ear, according to an aspect of the present disclosure.

### DETAILED DESCRIPTION

The following description sets forth exemplary aspects of the present disclosure. It should be recognized, however, that such a description is not intended as a limitation on the scope of the present disclosure. Rather, the description also encompasses combinations and modifications to those exemplary aspects described herein.

The present disclosure provides a system, device, and method for neuro-acoustic vagal modulation aimed at relieving stress and anxiety. The system and device utilize a dual-modality approach that combines non-invasive electrical stimulation with auditory neural entrainment to activate the parasympathetic nervous system. The electrical stimulation targets specific areas of the vagus nerve, namely the superior (jugular) ganglion and pharyngeal branches, while the auditory neural entrainment employs user-specific audio frequencies. The combination of the non-invasive electrical stimulation and the auditory neural entrainment by user-specific audio frequencies is an effective approach for relieving stress and anxiety of a user as the vagus nerve stimulation by the non-invasive electrical stimulation increases sensitivity of human auditory cortex thereby intensifying the effects of the audio frequencies as well as intended effect of the audio frequencies. Further, when the vagus nerve stimulation by the non-invasive electrical stimulation is combined with the auditory neural entrainment by user-specific audio frequencies, then the combination facilitates users in relieving stress and anxiety easily. The aforementioned combination thereby also increases the therapeutic effects of the non-invasive electrical stimulation.

The system and device are designed to be user-friendly and personalized. The device incorporates a smart patch that is placed behind the user's ear, over the mastoid bone. The patch houses gel electrodes and connects magnetically to an enclosure containing the electrical circuitry. The device can be switched on or off wirelessly and may be used in conjunction with earphones. Further, the aforementioned placement of the device has an advantageous effect of easy application when compared with the state of art devices for neuro-acoustic vagal modulation where the state of art device uses cumbersome gel before placement of a stimulator.

To ensure optimal stimulation and results, the system and device may employ a specialized model for accurate placement verification. The model uses accelerometer data to detect muscle twitches that may indicate incorrect placement of the device. If incorrect placement is detected, the user is guided to reposition the device correctly via a neuro-acoustic vagal modulation application.

The system and device also offer tailored stimulation sessions based on the user's needs. The user may select a session type via the neuro-acoustic vagal modulation application, and the system determines the appropriate stimulation parameters, including the stimulation waveform, session pattern, session duration, and audio signal characteristics.

Furthermore, the system and device incorporate a Galvanic Skin Response biofeedback unit that uses Galvanic Skin Response (GSR) data to optimize the stimulation intensity. The system creates a feedback loop that dynamically adjusts the stimulation intensity based on the user's real-time physiological responses.

In addition, the system integrates real-time data from other wearable devices, such as smartwatches and fitness trackers, to further optimize the timing and effectiveness of stimulation sessions. The system analyzes data such as heart rate variability (HRV), Electrodermal activity (EDA), heart rate, and activity levels, and adjusts the stimulation parameters accordingly.

Overall, the disclosed system, device, and method provide a unique and effective solution for stress and anxiety management, offering a personalized, adaptive, and comprehensive approach to parasympathetic nervous system activation.

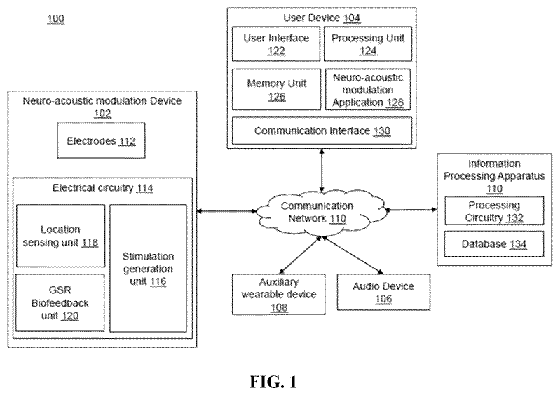

Referring to FIG. 1, the neuro-acoustic vagal modulation system 100 includes a neuro-acoustic vagal modulation device 102, a user device 104, an audio device 106, an auxiliary wearable device 108, and an information processing apparatus 110, all interconnected via a communication network 110.

The neuro-acoustic vagal modulation device 102 is designed to be positioned behind a user's ear and includes electrodes 112 and electrical circuitry 114. The electrodes 112 are gel electrodes that may be placed on a cloth with an adhesive layer to form a patch (not shown) and configured to deliver electrical stimulation signals to specific areas of the user's vagus nerve, namely the superior (jugular) ganglion and pharyngeal branches. The electrical circuitry 114 includes a stimulation generation unit 116, an location sensing unit 118, and a Galvanic Skin Response (GSR) biofeedback unit 120. The electrical circuitry 114 may be placed inside an enclosure (not shown) that may magnetically connect to the patch.

The stimulation generation unit 116 is coupled to the electrodes 112 and is configured to generate electrical stimulation signals based on stimulation parameters received from the information processing apparatus 110. In some aspects, the stimulation generation unit 116 may generate stimulation signals that are synchronized with audio signals delivered by the audio device 106, enhancing the overall neuromodulation effects.

The location sensing unit 118 is configured to detect the position of the neuro-acoustic vagal modulation device 102. In some cases, the location sensing unit 118 may use accelerometer data to detect muscle twitches that may indicate incorrect placement of the device 102. Specifically, the location sensing unit 118 may detect muscle twitches caused by the stimulation of muscle fibre rather than targeted vagus nerve fibre when the neuro-acoustic vagal modulation device 102 is not placed in an optimal area near the mastoid bone. A memory unit 126 may be configured to continuously collect accelerometer data corresponding to the muscle twitches sensed by the location sensing unit 118.

The accelerometer data is transmitted to a neuro-acoustic vagal modulation application 128 of the user device 104 and a processing unit 124 may process the data to determine if the neuro-acoustic vagal modulation device 102 is placed correctly. The processing unit 124 may determine the muscle activity threshold by using a combination of skin resistance, torque potential of stimulation, muscle activity score, and a user physiology constant. Specifically, the processing unit 124 may measure the muscle activity based on the following formula:

$$X ( Muscle Activity Threshold ) = ( {M}_{Activity} * {T}_{s} * {U}_{Calibration} ) / {R}_{Skin}$$

wherein, R_{Skin} (Skin Resistance) is in the range of 200 ohms to 1800 ohms, Ts (Torque susceptibility constant) is in the range of 0.1 to 0.84, M_{Activity} (Muscle Activity Score) is in the range of 0 to 100, and U_{Calibration} (User physiology constant) is in the range of 11 to 38. Interpretation of X:
X>1: Indicates incorrect device placement. The user should recheck and adjust the position of the device.
X<1: Indicates correct device placement.

Further, for measuring the muscle activity score, the processing unit 124 may combine accelerometer and gyroscope data from the memory unit 126 to determine the Muscle Activity Score (M_{Activity}). In operation, when a stimulation pulse at a controlled frequency (S_{f}) ranging between 10 Hz to 60 Hz is administered to the muscles, then the processing unit 124 may collect the accelerometer and the gyroscope data corresponding to the muscle response sensed by the location sensing unit 118, over a variable window of Δt milliseconds such that Δt value ranging from 1 ms to 20 ms.

Further, for each stimulation pulse i at time tᵢ, the processing unit 124 measures the muscle response corresponding to acceleration values along three axes (X, Y, Z) as aₓ(t), a_{y}(t), and a_{z}(t), and the muscle response corresponding to the angular velocity values along the three axes (X, Y, Z) as gₓ(t), g_{y}(t), and g_{z}(t). Furthermore, the processing unit 124 may calculate the Muscle Output (MO) by integrating the acceleration values and the angular velocities values into a single unit along with subtracting a baseline value (Bᵢ) from each of the acceleration values and the angular velocities values. The baseline value (Bᵢ) is a datum corresponding to accelerometer and gyroscope data that a processing unit may collect from the location sensing unit 108 before the administration of each stimulation pulse.

The processing unit 124 may calculate the Root Mean Square (RMS) of the accelerometer and gyroscope data over the window Δt using formula:

$$\sqrt{{{( \sqrt{\frac{1}{N} {\sum }_{i = 1 \vert ( {a}_{\text{?}} ( {t}_{i} ) - {B}_{x} ) + ( {a}_{y} ( {t}_{i} ) - {B}_{y} ) + {( {a}_{\text{?}} ( {t}_{i} ) - {B}_{\text{?}} )}^{\text{?}} \vert}^{N}} )}^{2} + \sqrt{\frac{1}{N} {\sum }_{i = 1}^{N} \vert ( {g}_{x} ( {t}_{i} ) - {B}_{yx} ) + ( {g}_{y} ( {t}_{i} ) - {B}_{yy} ) + {( {g}_{i} ( {t}_{i} ) - {B}_{yx} )}^{\text{?}} \vert} )}^{2}}$$

$$\text{?} \text{indicates text missing or illegible when filed}$$

Further, the processing unit 124 may calculate the Mean Average Deviation (MAD) of the accelerometer and gyroscope data by formula:

$${MAD}_{accel} = \frac{1}{N} {\sum }_{i = 1}^{N} \vert ( {a}_{\text{?}} ( {t}_{i} ) - {B}_{x} ) + ( {a}_{y} ( {t}_{i} ) - {B}_{y} ) + ( {a}_{\text{?}} ( {t}_{i} ) - {B}_{\text{?}} ) \vert$$

$${MAD}_{gyro} = \frac{1}{N} {\sum }_{i = 1}^{N} \vert ( {g}_{x} ( {t}_{i} ) - {B}_{yx} ) + ( {g}_{y} ( {t}_{i} ) - {B}_{yy} ) + ( {g}_{i} ( {t}_{i} ) - {B}_{yx} ) \vert$$

$$\text{?} \text{indicates text missing or illegible when filed}$$

Therefore, the combined MAD formula is:

$${MAD}_{combined} = \sqrt{{( {MAD}_{accel} )}^{2} + {( {MAD}_{gyro} )}^{2}}$$

Therefore, the final muscle activity is calculated by the processing unit by the formula:

$$MActivity = α \cdot {RMS}_{combined} + β \cdot {MAD}_{combined} ,$$

Where α and β are weighting factors to balance the contribution of RMS and MAD to the final muscle activity score.

For ensuring the correct placement of the neuro-acoustic vagal modulation device 102, the processing unit 124 may nullify the impact of torque susceptibility factors by determining the torque susceptibility factors (Tₛ) using the formula: Ts=1/(S_{I}*S_{F}*S_{Pw}), such that, S_{I} represents stimulation intensity constant that ranges between 0.06 to 0.23, S_{f} represents stimulation frequency constant that ranges between 10 to 60, and S_{pw} that represents stimulation pulse width constant that ranges between 2 to 7.

Further, the processing unit 124 may generate an error signal when the location sensing unit 118 is incorrectly placed. Further, the neuro-acoustic vagal modulation application 128 may display the error message based on the error signal and provide guidance to the user via a user interface 122 of the user device 104 for correct placement of the neuro-acoustic vagal modulation device 102. The proper placement of the neuro-acoustic vagal modulation device 102 facilitates the user to avoid unnecessary muscle stimulation and to achieve the desired therapeutic effects on the parasympathetic nervous system.

The GSR biofeedback unit 120 is configured to measure the user's galvanic skin response (GSR), providing real-time physiological response data. The data is transmitted to the information processing apparatus 110, which can dynamically adjust the stimulation parameters based on the received GSR data, ensuring personalized and effective therapy.

The user device 104 includes the user interface 122, the processing unit 124, the memory unit 126, the neuro-acoustic vagal modulation application 128, and a communication interface 130. The user interface 122 allows the user to interact with the system 100, including selecting a session type and receiving guidance for correct placement of the neuro-acoustic vagal modulation device 102. The processing unit 124 and memory unit 126 work together to process data and control the operation of the user device 104. The neuro-acoustic vagal modulation application 128 provides a user-friendly interface for interacting with the system 100, and the communication interface 130 facilitates data exchange with the neuro-acoustic vagal modulation device 102 and the information processing apparatus 110.

The information processing apparatus 110 includes processing circuitry 132 and a database 134. The processing circuitry 132 receives the session type selection from the user device 104, determines the stimulation parameters based on the selected session type, and transmits the stimulation parameters to the neuro-acoustic vagal modulation device 102. The processing circuitry 132 also receives position data and GSR data from the neuro-acoustic vagal modulation device 102, dynamically adjusts the stimulation parameters based on the received data, and transmits the adjusted stimulation parameters to the neuro-acoustic vagal modulation device 102. The database 134 stores relevant information for the operation of the system 100, including user data, session data, and physiological response data.

The communication network 110 facilitates data exchange between the neuro-acoustic vagal modulation device 102, the user device 104, the audio device 106, the auxiliary wearable device 108, and the information processing apparatus 110. In some aspects, the communication network 110 may be a wireless network, such as a Wi-Fi network, a cellular network, or a Bluetooth network. In other aspects, the communication network 110 may be a wired network, such as an Ethernet network. The specific type of communication network 110 used may depend on various factors, including the specific requirements of the system 100, the capabilities of the devices involved, and the user's preferences.

The GSR biofeedback unit 120 of the neuro-acoustic vagal modulation device 102 is configured to measure the user's GSR, providing real-time physiological response data. In some cases, the GSR biofeedback unit 120 may measure changes in the user's skin conductance, which can be indicative of changes in the user's stress or anxiety levels. The data is transmitted to the information processing apparatus 110, which can dynamically adjust the stimulation parameters based on the received GSR data, ensuring personalized and effective therapy.

The neuro-acoustic vagal modulation device 102 also includes a communication interface 130. The communication interface 130 is configured to communicate with the user device 104 and the information processing apparatus 110. In some aspects, the communication interface 130 may facilitate data exchange between the neuro-acoustic vagal modulation device 102 and other components of the system 100, enabling the device 102 to receive stimulation parameters and transmit position data and GSR data.

The electrical circuitry 114 of the neuro-acoustic vagal modulation device 102 is coupled to the stimulation generation unit 116, the location sensing unit 118, the GSR biofeedback unit 120, and the communication interface 130. The electrical circuitry 114 is configured to control the operation of these components. In some cases, the electrical circuitry 114 may receive stimulation parameters from the information processing apparatus 110, control the stimulation generation unit 116 to generate the electrical stimulation signals based on the received stimulation parameters, transmit position data from the location sensing unit 118 and GSR data from the GSR biofeedback unit 120 to the information processing apparatus 110, and receive adjusted stimulation parameters from the information processing apparatus 110 based on the transmitted position data and GSR data. This ensures that the stimulation delivered by the neuro-acoustic vagal modulation device 102 is tailored to the user's physiological state and the selected session type.

Referring to FIG. 1, the electrical circuitry 114 of the neuro-acoustic vagal modulation device 102 is configured to control the stimulation generation unit 116 to generate the electrical stimulation signals based on the stimulation parameters received from the information processing apparatus 110. In some aspects, the stimulation generation unit 116 may generate stimulation signals that are synchronized with audio signals delivered by the audio device 106, enhancing the overall neuromodulation effects. The synchronization of the electrical stimulation signals with the audio signals may be adjusted based on the received adjusted stimulation parameters, allowing for a more personalized and effective therapy.

The electrical circuitry 114 is also configured to transmit position data from the location sensing unit 118 and GSR data from the GSR biofeedback unit 120 to the information processing apparatus 110. The position data may include information about the location of the neuro-acoustic vagal modulation device 102, which can be used by the information processing apparatus 110 to verify the correct placement of the device 102. The GSR data may include information about the user's skin conductance, which can be indicative of the user's stress or anxiety levels. The real-time physiological response data allows the information processing apparatus 110 to dynamically adjust the stimulation parameters based on the user's current physiological state.

The electrical circuitry 114 is further configured to receive adjusted stimulation parameters from the information processing apparatus 110 based on the transmitted position data and GSR data. These adjusted stimulation parameters are used to control the stimulation generation unit 116 to generate the adjusted electrical stimulation signals. The ensures that the stimulation delivered by the neuro-acoustic vagal modulation device 102 is tailored to the user's physiological state and the selected session type.

The neuro-acoustic vagal modulation system 100 further comprises an audio device 106 configured to generate audio signals for acoustic modulation of brainwave frequencies. In some cases, the audio device 106 may generate audio signals that are synchronized with the electrical stimulation signals generated by the stimulation generation unit 116. The dual-modality approach of electrical stimulation and auditory neural entrainment enhances the overall neuromodulation effects, promoting relaxation and stress relief.

In some aspects, the electrical circuitry 114 may be further configured to synchronize the electrical stimulation signals with the audio signals. The synchronization may be adjusted based on the received adjusted stimulation parameters, allowing for a more personalized and effective therapy. For instance, if the user's GSR data indicates a high level of stress, the synchronization between the electrical stimulation signals and the audio signals may be adjusted to promote a more calming effect. Conversely, if the user's GSR data indicates a low level of stress, the synchronization may be adjusted to promote a more alert state. The dynamic adjustment of the synchronization between the electrical stimulation signals and the audio signals provides a highly personalized and adaptive approach to stress and anxiety management.

Referring to FIG. 1, the user device 104 of the neuro-acoustic vagal modulation system 100 includes the user interface 122, the processing unit 124, the memory unit 126, the neuro-acoustic vagal modulation application 128, and the communication interface 130. The user interface 122 allows the user to interact with the system 100, including selecting a session type and receiving guidance for correct placement of the neuro-acoustic vagal modulation device 102. The processing unit 124 and memory unit 126 work together to process data and control the operation of the user device 104. The neuro-acoustic vagal modulation application 128 provides a user-friendly interface for interacting with the system 100, and the communication interface 130 facilitates data exchange with the neuro-acoustic vagal modulation device 102 and the information processing apparatus 110.

In some aspects, the user interface 122 may be configured to receive a selection of a session type from the user. The session type may be selected from a group consisting of sleep, relax, and focus. The selected session type determines the stimulation parameters, which are then transmitted to the neuro-acoustic vagal modulation device 102 via the communication interface 130.

The audio device 106 of the neuro-acoustic vagal modulation system 100 is configured to deliver audio signals to the user. In some cases, the audio device 106 may be a pair of earphones, headphones, or any other suitable audio output device. The audio device 106 may generate audio signals that are synchronized with the electrical stimulation signals generated by the stimulation generation unit 116 of the neuro-acoustic vagal modulation device 102. The synchronization may be adjusted based on the adjusted stimulation parameters received, allowing for a more personalized and effective therapy.

The information processing apparatus 110 of the neuro-acoustic vagal modulation system 100 includes processing circuitry 132 and a database 134. The processing circuitry 132 is configured to receive the session type selection from the user device 104, determine the stimulation parameters based on the selected session type, and transmit the stimulation parameters to the neuro-acoustic vagal modulation device 102. The processing circuitry 132 is also configured to receive position data and GSR data from the neuro-acoustic vagal modulation device 102, dynamically adjust the stimulation parameters based on the received data, and transmit the adjusted stimulation parameters to the neuro-acoustic vagal modulation device 102. The database 134 stores relevant information for the operation of the system 100, including user data, session data, and physiological response data.

In some aspects, the processing circuitry 132 may be further configured to analyze post-session data to evaluate the effectiveness of the session and provide smart nudges to the user via the user device 104 suggesting additional sessions or professional support based on the analysis. The processing circuitry 132 may also receive data from the auxiliary wearable device 108 including at least one of heart rate, sleep time, wake-up time, activity levels, or heart rate variability (HRV), and adjust the stimulation parameters based on the received data from the auxiliary wearable device 108. In some cases, the processing circuitry 132 may calculate a Vagal Activity Index (VAI) based on the received data from the auxiliary wearable device 108 and the GSR data from the neuro-acoustic vagal modulation device 102, and optimize the stimulation parameters based on the calculated VAI. The processing circuitry 132 may also synchronize the electrical stimulation signals generated by the neuro-acoustic vagal modulation device 102 with the audio signals delivered by the audio device 106, and dynamically adjust the synchronization based on the calculated VAI and the received position data.

In some aspects of the present disclosure, the processing circuitry 132 of the information processing apparatus 110 employs advanced models and calculations to optimize the stimulation parameters. Specifically, the processing circuitry 132 calculates a Vagal Activity Index (VAI) using a complex formula that considers multiple physiological parameters. These parameters may include, but are not limited to, baseline heart rate during sleep (HR sleep), heart rate before sleep (HR_pre_sleep), heart rate during stimulation (HR stim), GSR data (GSR_current), circadian rhythm factors (CR_factor), recovery time from high activity (RT_recovery), and a user calibration constant (UCalibration).

The VAI formula may be expressed as follows:

$$VAI = α ( HR\_sleep ) + β ( HR\_pre \_sleep ) + γ ( HR\_stim ) + δ ( GSR\_current ) + ϵ ( CR\_factor ) + ζ ( RT\_recovery ) + UCalibration$$

Where α, β, γ, δ, ϵ, and ζ are weighting factors that balance the contribution of each component to the VAI, and UCalibration is a user physiology constant that accounts for individual variability in physiological responses.

The processing circuitry 132 uses the calculated VAI to adjust the stimulation parameters dynamically, ensuring that the stimulation delivered by the neuro-acoustic vagal modulation device 102 is tailored to the user's physiological state and the selected session type.

In some cases, the processing circuitry 132 may also consider circadian rhythm factors and recovery time from high activity when adjusting the stimulation parameters. For instance, the processing circuitry 132 may increase the stimulation intensity if the user's circadian rhythm data indicates that the user is in a high activity state, or decrease the stimulation intensity if the user's recovery time from high activity is short. The ensures that the stimulation delivered by the neuro-acoustic vagal modulation device 102 adapts to the user's individual physiological rhythms, providing a highly personalized and effective therapy.

In some aspects, the processing circuitry 132 may further adjust the synchronization between the electrical stimulation signals and the audio signals based on the calculated VAI and the received position data. The dynamic adjustment of the synchronization provides a highly personalized and adaptive approach to stress and anxiety management.

In some cases, the processing circuitry 132 may also use the calculated VAI to optimize the timing of the stimulation sessions. For instance, the processing circuitry 132 may schedule the stimulation sessions at times when the user's VAI indicates a high level of stress or anxiety, ensuring timely and effective intervention.

In some aspects, the processing circuitry 132 may also use the calculated VAI to provide smart nudges to the user via the user device 104. These smart nudges may suggest additional sessions or professional support based on the user's VAI, promoting consistent use of the system 100 and improving therapeutic outcomes.

In some cases, the processing circuitry 132 may also use the calculated VAI to evaluate the effectiveness of the stimulation sessions. By comparing the user's VAI before and after each session, the processing circuitry 132 can assess the impact of the session on the user's stress and anxiety levels, and adjust the stimulation parameters accordingly for future sessions.

In some aspects, the processing circuitry 132 may also use the calculated VAI to refine the models used by the system 100 over time. By analyzing the changes in the user's VAI over multiple sessions, the processing circuitry 132 can identify patterns and trends that can be used to improve the effectiveness of the stimulation sessions.

The continuous learning and adaptation make the neuro-acoustic vagal modulation system 100 a powerful tool for managing stress and anxiety in real-time.

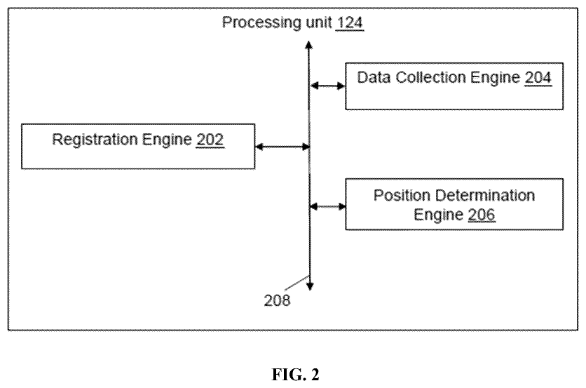

Referring to FIG. 2, the processing unit 124 of the user device 104 includes several components that work together to process data and determine device positioning. The registration engine 202, data collection engine 204, and position determination engine 206 are interconnected and communicate bidirectionally, allowing for data exchange and communication between the engines by way of a communication bus 208.

The registration engine 202 is configured to register the neuro-acoustic vagal modulation device 102 with the user device 104. In some aspects, the registration process may involve pairing the neuro-acoustic vagal modulation device 102 with the user device 104 via a communication network, such as a Bluetooth network. The registration engine 202 may also store information about the registered neuro-acoustic vagal modulation device 102 in the memory unit 126 of the user device 104. The information may include, for example, the device ID, user preferences, and session history.

The data collection engine 204 is configured to collect data from the neuro-acoustic vagal modulation device 102 and the auxiliary wearable device 108. The collected data may include, for example, position data from the location sensing unit 118 of the neuro-acoustic vagal modulation device 102, GSR data from the GSR biofeedback unit 120 of the neuro-acoustic vagal modulation device 102, and physiological data from the auxiliary wearable device 108. The collected data is processed by the processing unit 124 and stored in the memory unit 126 for further analysis and use.

The position determination engine 206 is configured to determine the position of the neuro-acoustic vagal modulation device 102 based on the collected position data. In some aspects, the position determination engine 206 may use an accelerometer-based model to analyze the position data and detect muscle twitches that may indicate incorrect placement of the neuro-acoustic vagal modulation device 102. If incorrect placement is detected, an error signal is generated and transmitted to the user interface 122. The user interface 122 then provides guidance to the user for correct placement of the neuro-acoustic vagal modulation device 102.

In some cases, the processing unit 124 may also include a stimulation control engine (not shown) that controls the generation and transmission of stimulation signals by the neuro-acoustic vagal modulation device 102. The stimulation control engine may receive stimulation parameters from the information processing apparatus 110, control the stimulation generation unit 116 of the neuro-acoustic vagal modulation device 102 to generate the stimulation signals based on the received stimulation parameters, and transmit the stimulation signals to the neuro-acoustic vagal modulation device 102 via the communication interface 130.

In some aspects, the processing unit 124 may further include a session selection engine (not shown) that allows the user to select a session type via the user interface 122. The session selection engine may transmit the selected session type to the information processing apparatus 110, which determines the stimulation parameters based on the selected session type and transmits the stimulation parameters to the neuro-acoustic vagal modulation device 102 via the communication network 110.

In some cases, the processing unit 124 may also include a biofeedback analysis engine (not shown) that analyzes the collected GSR data and physiological data to assess the user's physiological response to the stimulation. The biofeedback analysis engine may dynamically adjust the stimulation parameters based on the analyzed data and transmit the adjusted stimulation parameters to the neuro-acoustic vagal modulation device 102 via the communication interface 130. The ensures that the stimulation delivered by the neuro-acoustic vagal modulation device 102 is tailored to the user's physiological state and the selected session type.

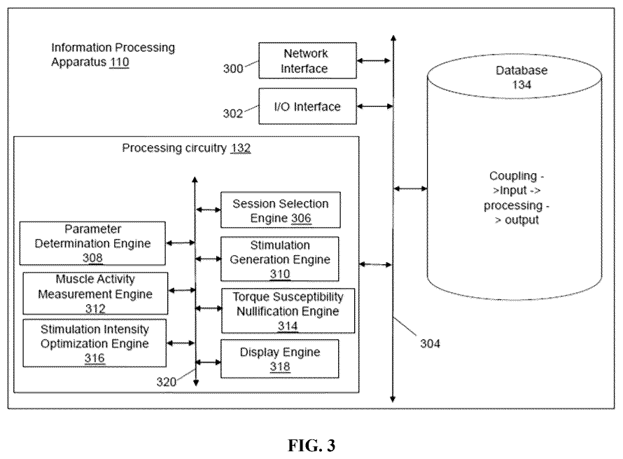

Referring to FIG. 3, the information processing apparatus 110 of the neuro-acoustic vagal modulation system 100 includes processing circuitry 132 and a database 134. The processing circuitry 132 comprises several interconnected components, including a session selection engine 306, a parameter determination engine 308, a stimulation generation engine 310, a muscle activity measurement engine 312, a torque susceptibility nullification engine 314, a stimulation intensity optimization engine 316, and a display engine 318 communicatively coupled to each other by way of a communication bus 320. These components work together to process data, determine stimulation parameters, generate stimulation signals, and analyze post-session data for the neuro-acoustic vagal modulation system 100.

The information processing apparatus 110 of the neuro-acoustic vagal modulation system 100 incorporates several specialized engines within its processing circuitry 132, each designed to handle specific aspects of the neuromodulation process. The session selection engine 306 interfaces with the user device 104 to receive the user's chosen session type, which can be sleep, relax, or focus.

Upon receiving the session type, the parameter determination engine 308 employs a sophisticated model to establish the optimal stimulation parameters. The model considers not only the selected session type but also the user's current physiological state and historical data stored in the database 134. The resulting parameters encompass various aspects of the stimulation, including waveform characteristics, session patterns, duration, and complementary audio signal properties.

The stimulation generation engine 310 then utilizes these parameters to create precise stimulation signals. These signals are meticulously crafted to synchronize with the audio signals produced by the audio device 106, creating a harmonized neuromodulation effect that targets both the vagus nerve and auditory pathways simultaneously.

To ensure optimal device placement and effectiveness, the muscle activity measurement engine 312 analyzes accelerometer data from the neuro-acoustic vagal modulation device 102. The engine quantifies muscle activity, which serves as a key indicator for correct device positioning and helps in fine-tuning stimulation parameters based on the user's real-time physiological responses.

The torque susceptibility nullification engine 314 plays a crucial role in maintaining the accuracy of the Muscle Activity Threshold (X). By accounting for and neutralizing torque susceptibility factors, the engine ensures that the neuro-acoustic vagal modulation device 102 remains optimally positioned throughout the session, maximizing the therapeutic benefits of vagus nerve stimulation and auditory neural entrainment. Working in tandem with these engines, the stimulation intensity optimization engine 316 continuously monitors the user's Galvanic Skin Response (GSR) data. The real-time biofeedback allows for dynamic adjustments to the stimulation intensity, ensuring that the therapy remains both comfortable and effective throughout the session, adapting to subtle changes in the user's physiological state.

The display engine 318 serves as the visual interface between the system and the user, providing clear guidance on device placement, session information, and real-time physiological data through the user device 104. The visual feedback enhances user engagement and promotes proper use of the system.

Post-session analysis is a key feature of the processing circuitry 132. By evaluating changes in various physiological markers such as heart rate variability (HRV), Electrodermal activity (EDA), heart rate, and activity levels, the system can assess the effectiveness of each session. The analysis informs the system's smart nudge feature, which provides personalized recommendations for future sessions or suggests professional support when appropriate, thereby continually optimizing the user's experience and therapeutic outcomes.

The database 134, intricately linked to the processing circuitry 132, serves as the system's memory, storing and managing a wealth of user-specific data. The includes detailed session histories, physiological response patterns, and post-session analyses. The robust data management capabilities of the database 134 enable the system to refine its models over time, leading to increasingly personalized and effective neuromodulation therapies for each user.

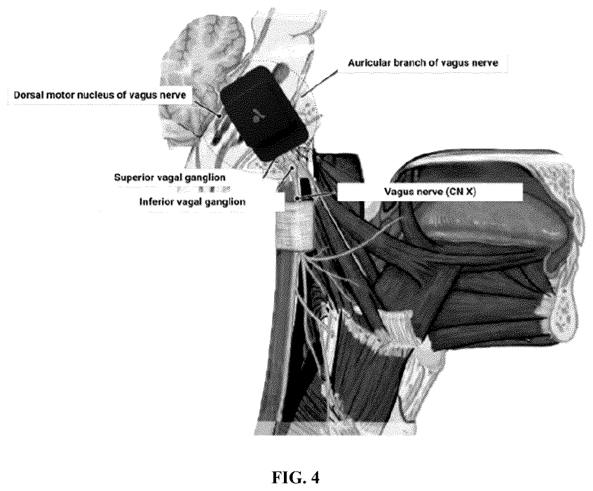

FIG. 4 illustrates a correctly placed neuro-acoustic vagal modulation device 102 to stimulate the auricular branch and the pharyngeal branch of the vagus nerves, according to an aspect of the present disclosure. As illustrated in the FIG. 4, the neuro-acoustic vagal modulation device 102 is placed behind the ear over the mastoid bone for stimulating the auricular branch as well as cervical branch and the pharyngeal branch of the vagus nerve for activation of the parasympathetic nervous system.

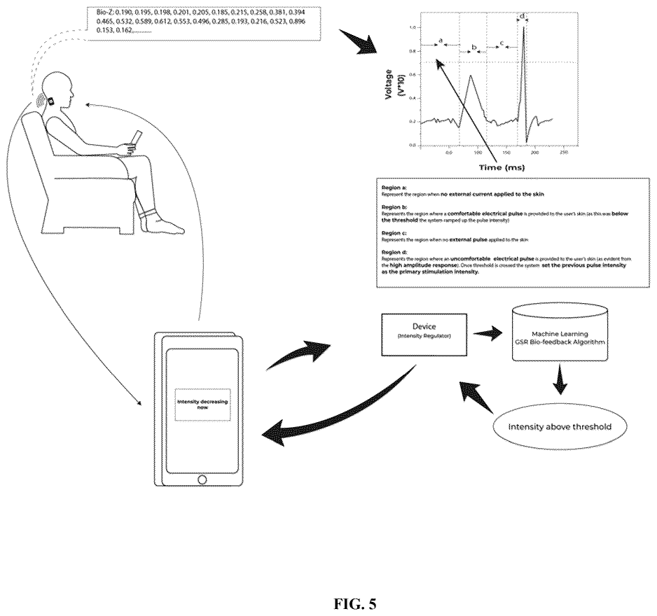

FIG. 5 illustrates a representation of working of the GSR bio-feedback mechanism, according to an aspect of the present disclosure. As illustrated in FIG. 5, after correctly placing the neuro-acoustic vagal modulation device 102 behind the ear over the mastoid bone, the processing circuitry 132 may monitor the galvanic skin response. The processing circuitry 132 may provide the electrical stimulation pulses to the user by way of the neuro-acoustic vagal modulation device 102 and monitor the GSR of a user corresponding to the electrical stimulation pulses provided. The processing circuitry 132 may thereby determine the right electrical stimulation pulse intensity for a specific user. Further, the processing circuitry 132 may gradually increase the intensity of electrical stimulation pulse and continually monitor the GSR corresponding to the increment in the intensity of electrical stimulation pulse, thereby allowing the user to get maximum therapeutic benefit of the electrical stimulation. Further, after providing a specific electrical stimulation pulse of specific intensity, the processing circuitry 132 by using the data provided from the auxiliary wearable device 108 and sensors in GSR biofeedback unit 120, monitors the response of the body of the user with respect to the intensity of the electrical stimulation provided to the user i.e., the amount of time a user is calm, relieved etc. Furthermore, based on the response of the body of the user, the processing circuitry may provide a score to that represents the status of the parasympathetic nervous system or vagal tone of the body of the user. In some aspects, the processing circuitry may also provide a schedule that a user may follow to improve the score thereby increasing the stress resilience of the user.

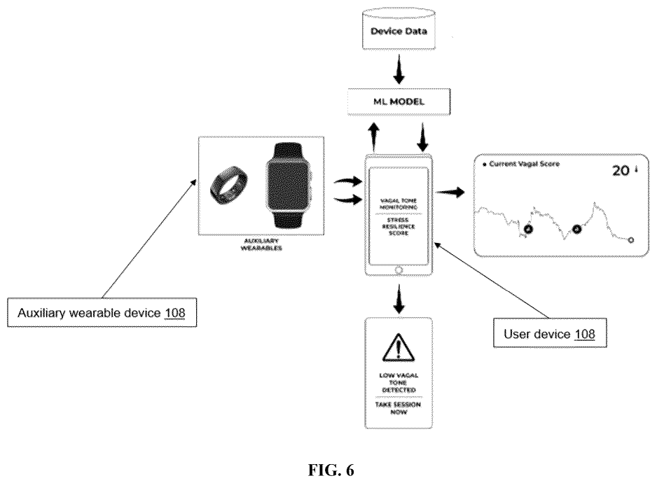

FIG. 6 illustrates a representation of working of the neuro-acoustic vagal modulation system, according to an aspect of the present disclosure. As illustrated in FIG. 6, when the neuro-acoustic vagal modulation device 102 is correctly placed, the processing circuitry 132 by way of the neuro-acoustic vagal modulation device 102, receives the user's baseline GSR in a relaxed state to establish a reference point. In some aspects of the present disclosure, the processing circuitry 132 may obtain the baseline measurement when the user is present in a controlled, relaxed environment such that GSR sensors present in the GSR biofeedback unit 120 record the skin conductance over a period of five minutes. The processing circuitry 132 may calculate average values of the baseline measurements and may therefore set the baseline GSR. Further, the processing circuitry 132 may monitor the GSR data of the user during the stimulation session and processes the GSR data to dynamically adjust the stimulation intensity. Specifically, the processing circuitry 132 may create a feedback loop that ensures the intensity of stimulation adapts to the user's physiological state. To optimize the stimulation intensity, the processing circuitry 132 may set predefined upper and lower GSR thresholds relative to the baseline measurement. Further, the processing circuitry may increase the stimulation intensity when the monitored GSR falls below the lower threshold indicating relaxation. On the other hand, the processing circuitry 132 may decrease the stimulation intensity when the monitored GSR exceeds the upper threshold indicating stress. The processing circuitry 132 may maintain the stimulation intensity when the monitored GSR remains within the optimal range. In some other aspects of the present disclosure, the processing circuitry 132 may allow the user to override the aforementioned automatic adjustments by way of the neuro-acoustic vagal modulation application 128.

The processing circuitry may optimize the stimulation intensity (I) using the formula:

$$( d ( {GSR}_{currentPeakAvg} - {GSR}_{baselinePeakAvg} ) / dt ) < 0.02 * {U}_{cal} * {R}_{skin} ( {S}_{i} < {S}_{oi} )$$

$$( d ( {GSR}_{currentPeakAvg} - {GSR}_{baselinePeakAvg} ) / dt ) \approx 0.02 * {U}_{cal} * {R}_{skin} ( Si = Soi , optimal intensity reached )$$

$$( d ( {GSR}_{currentPeakAvg} - {GSR}_{baselinePeakAvg} ) / dt ) > 0.02 * {U}_{cal} * {R}_{skin} ( Si > Soi ) ,$$

such that GSR_baseline is the baseline skin conductance measured during initial calibration, GSR_current is the real-time skin conductance measured during the stimulation session, and UCalibration is a user physiology constant to account for individual variability in skin conductance responses.

Furthermore, as illustrated in FIG. 6, the neuro-acoustic vagal modulation system 100 may employ a feedback loop that integrates real-time data from auxiliary wearable device 108 such as smartwatches, fitness trackers or the like. The neuro-acoustic vagal modulation system 100 may thereby use data such as heart rate variability (HRV), Electrodermal activity (EDA), heart rate, or the like to optimize the timing and effectiveness of stimulation sessions. The neuro-acoustic system 100 may furthermore analyze the aforementioned data to gain insights into the user's vagal nerve activity and overall autonomic nervous system health.

In some embodiments of the present disclosure, the processing circuitry 132 may monitor the vegal tone of the user by assessing the baseline activity of the user's vagal nerve via tracking the heart rate during sleep, before sleep, and during stimulation sessions. Specifically, the processing circuitry 132 may receive the heart rate data of the user during sleep and establishes a baseline of the user's vagal tone. When the user takes the stimulation session, the processing circuitry may capture the vagal response of the user and compare the vagal response with the baseline data to evaluate the effectiveness of the stimulation.

The neuro-acoustic system 100 may further consider factors such as circadian rhythm and the time taken by the individual's body to return to a normal state from a high activity state.

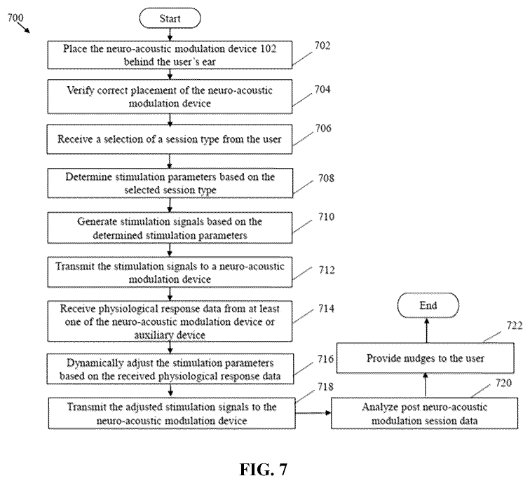

Referring to FIG. 7, the method for neuromodulation begins with step 702, where the user places the neuro-acoustic vagal modulation device 102 behind their ear. The device 102 is designed to be positioned over the mastoid bone, ensuring optimal targeting of the superior ganglion and pharyngeal branches of the vagus nerve. In some aspects, the device 102 may include an adhesive layer to secure the device in place during the session.

In step 704, the neuro-acoustic vagal modulation device 102 is connected to the user device 104. The connection may be established wirelessly via the communication interface 130, which may utilize a variety of communication protocols, including but not limited to Bluetooth, Wi-Fi, or cellular networks. Once the connection is established, the neuro-acoustic vagal modulation device 102 automatically connects to the neuro-acoustic vagal modulation application 128 on the user device 104.

Further, when the connection is established, the neuro-acoustic vagal modulation application 128 checks for correct placement of the neuro-acoustic vagal modulation device 102 using a proprietary accelerometer-assisted model. The model analyzes accelerometer data from the sensing unit 118 of the device 102 to detect muscle twitches that may indicate incorrect placement of the device 102. If incorrect placement is detected, an error is raised in the application 128, which then guides the user to place the device correctly. The ensures optimal stimulation positioning and maximizes the benefits of vagus nerve stimulation and auditory neural entrainment.

In step 706, after proper placement, the application 128 prompts the user to select a session type. The session type may be selected from a group consisting of sleep, relax, and focus. The selected session type determines the stimulation parameters, which are then transmitted to the neuro-acoustic modulation device 102 via the communication interface 130.

In step 708, the processing circuitry 132 may be configured to determine stimulation parameters based on the selected session type.

In step 710, the stimulation generation unit 116 of the neuro-acoustic modulation device 102 generates stimulation signals based on the determined stimulation parameters by way of the processing circuitry 132. The stimulation signals are designed to stimulate the superior ganglion and pharyngeal branches of the vagus nerve, leading to an on-demand activation of the parasympathetic nervous system.

To enhance the effectiveness of the stimulation session, an audio signal is paired with the electrical stimulation. The audio signal may be delivered to the user via the audio device 106, which may be a pair of earphones, headphones, or any other suitable audio output device. The audio signal is synchronized with the electrical stimulation signals, creating a profound sense of relaxation and calmness.

In step 712, the stimulation signals generated along with the audio signal may be transmitted to the neuro-acoustic vagal modulation device 102 positioned behind the user's ear.

In step 714, the physiological response data from at least one of the neuro-acoustic vagal modulation device 102 or an auxiliary wearable device 108 maybe received by way of the processing circuitry 132. Specifically, during the stimulation session, the neuro-acoustic vagal modulation device 102 by way of the processing circuitry 132 may continuously monitors the user's physiological responses. The GSR biofeedback unit 120 of the device 102 measures the user's GSR levels in real-time, providing valuable physiological response data. In some aspects, the device 102 may also receive physiological response data from the auxiliary wearable device 108, which may include data such as heart rate variability (HRV), Electrodermal activity (EDA), heart rate, and activity levels.

In Step 716, the processing circuitry 132 of the information processing apparatus 110 may dynamically adjust the stimulation parameters based on the received physiological response data. The adjusted stimulation parameters are then transmitted to the neuro-acoustic vagal modulation device 102 via the communication network 110, ensuring that the stimulation intensity adapts to the user's physiological state.

In step 718, the processing circuitry 132 may transmit the adjusted stimulation signals to the neuro-acoustic vagal modulation device (102) for application to the user.

Further, the stimulation session continues for a predetermined duration. The session duration is predefined based on the selected session type, after which the user can continue their daily activities. The duration of the session may vary depending on the user's needs and the selected session type.

Following the session, in step 720, the processing circuitry 132 of the information processing apparatus 110 analyzes the post-session data. The analysis evaluates the effectiveness of the session and optimizes future sessions. The post-session data may include changes in the user's physiological responses, such as GSR, HRV, heart rate, and activity levels.

In step 722, the processing circuitry 132 provides smart nudges to the user via the user device 104 based on the post-session analysis. These smart nudges may suggest additional sessions or professional support as needed. For instance, if the user's heart rate is too high for an extended period of time, the system 100 may prompt the user to take a relaxation session. If multiple sessions are detected over a short period of time, the system 100 may prompt the user to connect with a psychiatrist or a mental health coach. These smart nudges help to improve therapeutic outcomes and promote consistent use of the system 100.

Thus, the system, device, and method provide several technical advantages:
1. Enhanced personalization through real-time biofeedback integration, allowing dynamic adjustment of stimulation parameters based on individual physiological responses.
2. Improved accuracy and effectiveness of stimulation through precise placement verification using an accelerometer-based model, ensuring optimal targeting of the superior ganglion and pharyngeal branches of the vagus nerve.
3. Synergistic combination of electrical stimulation and acoustic modulation, leveraging the benefits of both modalities to achieve more comprehensive neuromodulation effects.
4. Advanced adaptability through integration with auxiliary wearable devices, enabling the system to consider a wider range of physiological data for optimizing stimulation parameters.
5. Increased user engagement and therapeutic outcomes through smart nudges and post-session analysis, promoting consistent use and timely professional intervention when needed.
7. Sophisticated vagal tone measurement and optimization through the calculation of a Vagal Activity Index, allowing for more precise tuning of stimulation parameters to individual user needs.
8. The small size of the neuro-acoustic vagal modulation device (i.e., 25 mm by 25 mm) allows a user to easily carry the device and use the device in case of emergencies such as suddenly having a bout of stress.

The neuro-acoustic vagal modulation system ensures user safety by making gradual adjustments in the stimulation intensity and enhances the efficacy of the neuro-acoustic vagal modulation therapy by tailoring the stimulation intensity to individual physiological responses.

The neuro-acoustic vagal modulation system represents a significant technical advancement in non-invasive stress and anxiety relief by providing a highly personalized, adaptive, and comprehensive approach to parasympathetic nervous system activation.

A number of implementations have been described. Nevertheless, it will be understood that various modifications may be made without departing from the scope of the disclosure. Accordingly, other implementations are within the scope of the following claims.

1. A neuro-acoustic vagal modulation system (100) comprising:
a neuro-acoustic vagal modulation device (102) comprising:
electrodes (112) configured to be positioned behind a user's ear;
a stimulation generation unit (116) coupled to the electrodes (112) and configured to generate electrical stimulation signals;
a location sensing unit (118) configured to detect a position of the device;
a galvanic skin response (GSR) biofeedback unit (120) configured to measure the user's GSR;
a user device (104) configured to enable the user in accurate placement of the electrodes (112) behind the user's ear;
an auxiliary wearable device (108) that is configured to determine data comprising one of, heart rate, sleep time, wake-up time, activity levels, heart rate variability (HRV), or a combination thereof;
an audio device (106) configured to deliver audio signals to the user; and
processing circuitry (132) that is coupled to the neuro-acoustic vagal modulation device (102), the user device (104), the auxiliary wearable device (108), and the audio device (106), and configured to:
receive a session type selection from the user device (104);
determine initial stimulation parameters based on the selected session type;
generate initial stimulation signals based on the initial stimulation parameters;
transmit the initial stimulation signals to the neuro-acoustic vagal modulation device (102);
receive position data and GSR data from the neuro-acoustic vagal modulation device (102);
receive data from the auxiliary wearable device (108);
calculate a Vagal Activity Index (VAI) based on the received data from the auxiliary wearable device (108) and the GSR data from the neuro-acoustic vagal modulation device (102); and dynamically adjust the initial stimulation parameters based on the received position data and VAI; and
transmit the adjusted stimulation parameters to the neuro-acoustic vagal modulation device (102).

2. The neuro-acoustic vagal modulation system (100) as claimed in claim 1, wherein the processing circuitry (132) is further configured to:
analyze post-session data to evaluate effectiveness of the session; and
provide smart nudges to the user via the user device (104) suggesting additional sessions or professional support based on the analysis.

3. The neuro-acoustic vagal modulation system (100) as claimed in claim 1, wherein the processing circuitry (132) is further configured to:
synchronize the electrical stimulation signals generated by the neuro-acoustic vagal modulation device (102) with the audio signals delivered by the audio device (106); and
dynamically adjust the synchronization based on the calculated VAI and the received position data.

4. A method (700) for neuro-acoustic vagal modulation, comprising:
receiving (706), by way of processing circuitry (132), a selection of a session type from a user device (104), wherein the processing circuitry (132) is coupled to a neuro-acoustic vagal modulation device (102), the user device (104), an auxiliary wearable device (108), and an audio device (106);
determining (708), by way of the processing circuitry (132), initial stimulation parameters based on the selected session type;
generating (710), by way of the processing circuitry (132), initial stimulation signals based on the determined initial stimulation parameters;
transmitting (712), by way of the processing circuitry (132), the initial stimulation signals to the neuro-acoustic vagal modulation device (102) positioned behind a user's ear;
receiving (714), by way of the processing circuitry (132), position data of the neuro-acoustic vagal modulation device (102), galvanic skin response (GSR) data from a GSR biofeedback unit (120) of the neuro-acoustic vagal modulation device (102), and data from the auxiliary wearable device (108), wherein the data from the auxiliary wearable device (108) comprising one of, heart rate, sleep time, wake-up time, activity levels, heart rate variability (HRV), or a combination thereof;
dynamically adjusting (716), by way of the processing circuitry (132), the initial stimulation parameters based on the received physiological response data; and
transmitting (718), by way of the processing circuitry (132), the adjusted stimulation signals to the neuro-acoustic vagal modulation device (102) for application to the user.

5. The method (700) as claimed in claim 4, wherein the session type is selected from a group consisting of sleep, relax, and focus.

6. The method (700) as claimed in claim 4, prior to the selection of the session type, the method (400) further comprising:
verifying (704), by way of the processing unit (124) of the user device (104), correct placement of the neuro-acoustic vagal modulation device (102) using an accelerometer-based technique.

7. The method as claimed in claim 4, further comprising:
analyzing (720), by way of the processing circuitry (132), post neuro-acoustic vagal modulation session data; and
providing (722), by way of the processing circuitry (132), nudges to the user.
